(19) United States
(12) Patent Application Publication
(10) Pub. No.: US 2014/0271273 A1
(43) Pub. Date: Sep. 18, 2014

(54) HANDHELD OCULAR ASPIRATION TOOL

(71) Applicant: Novartis AG, Basel (CH)

(72) Inventor: John Richard Carpenter, Irvine, CA (US)

(73) Assignee: Novartis AG, Basel (CH)

(21) Appl. No.: 14/203,753

(22) Filed: Mar. 11, 2014

Related U.S. Application Data
(60) Provisional application No. 61/793,987, filed on Mar. 15, 2013.

(51) Int. Cl. A61M 1/00; F04B 45/08; F04B 43/12

(52) U.S. Cl. CPC A61M 1/0058; F04B 43/1253; F04B 45/08
USPC 417/412

(57) ABSTRACT

A variety of systems, processes, and techniques may provide ocular aspiration. In certain implementations, a handheld ocular aspiration tool may include an electrical motor, a peristaltic pump assembly, a fluid input port, and a fluid output port. The peristaltic pump assembly may include a helically wound compressible conduit and a plurality of variable radius rollers. The rollers may be configured to increasingly occlude the conduit as the rollers are rotated in contact with the conduit, wherein two rollers substantially occlude the conduit at one time to pump fluid through the conduit. The fluid input port may be in fluid communication with an input end of the peristaltic pump assembly, and the fluid output port may be in fluid communication with an output end of the peristaltic pump assembly.

### CROSS-REFERENCE TO RELATED APPLICATIONS

This application claims the benefit of U.S. Provisional Application No. 61/793,987, filed Mar. 15, 2013, the entire contents of which are incorporated herein by reference.

### BACKGROUND

This disclosure relates to the field of ocular surgery, and more particularly to an aspiration tool for ocular surgery.

The human eye, in simple terms, functions to provide vision by transmitting and refracting light through a clear outer portion called the cornea and focusing the image by way of the lens onto the retina at the back of the eye. The quality of the focused image depends on many factors including the size, shape, and length of the eye, and the shape and transparency of the cornea and lens.

When trauma, age, or disease causes the lens to become less transparent, vision deteriorates because of a reduction in light transmitted to the retina. This deficiency in the eye's lens is medically known as a cataract. The treatment for this condition is often surgical removal of the lens and implantation of an artificial lens, often termed an intraocular lens (IOL).

An IOL is often foldable and inserted into the eye through a relatively small incision by being advanced through an insertion cartridge, which causes the IOL to fold. The IOL is typically advanced through the insertion cartridge by a plunger-like device.

Before inserting an IOL, the old lens is usually removed through a process called phacoemulsification. In phacoemulsification, an eye's lens is emulsified with an ultrasonic hand piece and aspirated from the eye. Aspirated fluids are replaced with an irrigation of water/saline solution, thus maintaining the shape of the anterior chamber. The irrigation fluid and the aspiration suction are usually supplied by a remote surgical console, which is coupled to the hand piece through several feet of tubing.

Typically, a first stage extracts the main portions of the lens, while a second stage is removes any remaining pieces of the lens. An irrigation-aspiration probe may be used to aspirate any remaining cortical matter, while leaving the posterior capsule intact.

### SUMMARY

Various systems, processes, and techniques are disclosed for an ocular aspiration. In one aspect, a handheld ocular aspiration tool may include an electrical motor and a peristaltic pump assembly. The pump assembly may include a helically wound compressible conduit and at least one variable radius roller. The at least one variable radius roller may be configured to increasingly occlude the conduit as the roller is rotated in contact with the conduit. The at least one variable radius roller may be operable to substantially occlude the conduit at one time to pump fluid through the conduit.

Another aspect may include a peristaltic pump for ocular surgery. The peristaltic pump may include an armature, a plurality of rollers rotatably coupled to the armature, a flexible conduit, and a casing surrounding the armature and the plurality of rollers. Each of the plurality of rollers may include a first radius portion and a second radius portion. The casing comprising a helical groove configured to receive the flexible conduit and guide the flexible conduit around the armature and the plurality of rollers, the helical groove positioning the conduit such that the flexible conduit is in a substantially open condition when contacted by the first radius portion of a roller of the plurality of rollers in a first location of the helical groove and in a substantially occluded condition when contacted by the second radius portion of the roller of the plurality of rollers in a second location of the helical groove.

The various aspects may include one or more of the following features. The at least one radius roller may include a plurality of variable radius rollers. Two variable radius rollers of the plurality of variable radius rollers may substantially occlude the conduit at one time to pump fluid through the conduit. Each of the plurality of variable radius rollers may include a variable radius section and a substantially constant radius section. The variable radius section may be operable to gradually occlude the helically wound compressible conduit as each of the plurality of variable radius rollers is rotated in contact with the helically wound compressible conduit until the helically wound compressible conduit is substantially occluded. The constant radius section is operable to maintain the occlusion as each of the plurality of variable radius rollers is continued to be rotated in contact with the helically wound compressible conduit

The various aspects may also include one or more of the following features. The peristaltic pump assembly may also include a casing. An inner wall of the casing may provide a helical groove for receiving the helically wound compressible conduit. The peristaltic pump assembly may include an armature. The plurality of variable radius rollers may be rotationally coupled to the armature. The motor may be operable to drive the armature. A variable radius of the plurality of variable radius rollers may have a slope of approximately 15 degrees.

The various aspects may also include one or more of the following features. The first radius portion may taper from a first radius to a second radius larger than the first radius. The first radius portion may be configured to engage the conduit in a substantially unoccluded condition at the first radius and progressively occlude the conduit a location where the first radius portion engages the conduit moves from the first radius to the second radius. A gradient of or slope defined by the taper of the first radius portion may be approximately 15 degrees. The second radius portion may include a constant radius. The conduit may be substantially occluded when in contact with the second radius portion.

Various other features will be apparent to those skilled in the art from the following detailed description and claims, as well as the accompanying drawings.

### BRIEF DESCRIPTION OF THE DRAWINGS

FIG. 1A is a partial cutaway perspective view of an example peristaltic pump showing changes in the cross-section of a flexible conduit at various locations therealong.

FIG. 1B is a partial cross-sectional view of the example peristaltic pump shown in FIG. 1A.

FIG. 2A is a cross-sectional view of an example pump assembly.

FIG. 2B is a partial cross-sectional view of an example peristaltic pump of the pump assembly of FIG. 2A.

FIG. 2C shows an example armature and rollers of the example peristaltic pump shown in FIGS. 2A and 2B.

FIG. 2D is a perspective view of the example peristaltic pump shown in FIGS. 2A, 2B, and 2C.

FIG. 3 is a perspective view of an example handheld ocular aspiration tool.

FIG. 4 is a cross-section view of an example irrigation-aspiration unit for use with an ocular aspiration tool.

### DETAILED DESCRIPTION

FIGS. 1A-1B illustrate an example peristaltic pump 100. Peristaltic pump 100 may, for example, be used in a handheld ocular aspiration tool, an example of which will be discussed below.

Peristaltic pump 100 includes a motor shaft 120 configured to mechanically interface with a motor adapted to drive motor shaft 120 in a rotary manner. Motor shaft 120 is also configured to turn an armature 150, which could, for example, be made of thermal plastic (e.g., polycarbonate). Three variable radius rollers 110a, 110b, 110c are pivotally coupled to armature 150 at apertures 160. Rollers 110a, 110b, 110c may, for example, also be made of thermal plastic (e.g., polycarbonate). Pinning loci 160 permit rollers 110a, 110b, 110c to rotate relative to armature 150. Although the example peristaltic pump 100 is illustrated as including three rollers 110a, 110b, 110c, the scope of the disclosure is not so limited. Rather, in other implementations, the peristaltic pump 100 may include more than three rollers. In still other implementations, the peristaltic pump may 100 include fewer than three rollers. For example, in some implementations, as few as a single roller may be used and operable to pump fluid.

Peristaltic pump 100 also includes a casing 170 that surrounds the peristaltic pump. Casing 170 may, for example, be made of thermal plastic (e.g., polycarbonate). In other implementations, the casing 170 may be made of other materials. For example, in some implementations, the casing 170 may be formed from a metal, such as stainless steel or titanium. In still other instances, the casing 170 may be formed from any suitable material.

Casing 170 is configured to permit a flexible conduit 130 to fit securely between rollers 110a, 110b, 110c and casing 170. As seen in FIG. 1B, casing 170 includes a helical guide groove 140 formed in an interior surface 172 of the casing 170. The helical guide groove 140 is configured to the conduit 130 around casing 170. The conduit 130 may, for example, be a hose, tube, or other conduit and may be made of a flexible material. For example, the conduit 130 may be formed from an elastomer (e.g., silicone rubber) or a thermoplastic elastomer. In other implementations, the conduit 130 may be formed from any suitable material. In operation, fluid enters peristaltic pump 200 through conduit 130 at an input end A of peristaltic pump 100 and exits at an output end E of peristaltic pump 100.

Although three rollers 110a, 110b, 110c are shown in FIG. 1, in other implementations, a pump may include a single roller and be operable to pump fluid. For example, a pump having a helically wound conduit such that there is sufficient overlap between coils of the helical conduit to allow the roller to trap a volume of fluid between the locations of the conduit compress by the roller is operable to pump fluid and is within the scope of this disclosure.

As can be seen more distinctly in FIG. 1B, armature 150 includes first and second flanges 152. The flanges 152 include a plurality of tabs 156. The tabs 156 include the apertures 160 at which the rollers 110a, 110b, 110c are coupled. The apertures 160 receive pins 162. In some instances, the pins 162 may be integrally formed with the rollers 110a, 110b, and 110c. In other instances, the pins 162 may be separate from the rollers 110a, 110b, 110c, and the pins may extend therethrough.

As illustrated in FIG. 1B, the rollers 110a, 110b, 110c may include a first zone 112 and a second zone 114. The first zone 112 may include a variable radius. Particularly, the first zone 112 may have a tapered shape. The second zone 114 may include a substantially constant radius. In the illustrated implementation, first zone 112 may have a slope of approximately 15 degrees compared to the radius of second zone 114. Other slopes may be used in other implementations. Other implementations may have other configurations. For example, a roller may have a single zone with a constant gradient, a gradual transition from a gradient to a substantially constant-radius surface, or an irregular gradient.

In operation, rollers 110a, 110b, 110c compress and release conduit 130 as they rotate around the inside of casing 130 on armature 150. Points B, C, and D indicate a cross-sectional shape of the conduit 130 at those respective locations. Particularly, those points reflect the deformational shape of those locations of the conduit 130 and illustrate the amount by which the passage within the conduit 130 is obstructed by compression of the conduit 130 by the rollers 110a, 110b, and 110c. Near input end A, a portion of conduit 230 is being contacted by the first zone 112 of roller 110c, which has a reduced radius value at the point of contact. In this case, conduit 130 is in a substantially open state at the point of contact, and fluid may be flowing into the conduit at this point. As armature 150 advances rollers 110a, 100b, 110c around casing 170, occlusion of the conduit 130 increases smoothly due to the variable radius of the rollers 110a, 110b, 110c. For example, at point B, approximately half of conduit 130 is being occluded by the second zone 114 of roller 110a while the other half is being only partially occluded by the first zone 112 of the roller 110a. The conduit 130 is progressively occluded as a result of the variable radius rollers 110a, 110b, 110c and the helical configuration of the conduit 130 along the interior surface 172 of the casing 170. As the rollers 110a, 110b, 110c are rotated in the armature 150 relative to the conduit 130, a location at which any one of the rollers 110a, 110b, 110c is in contact with the conduit 130 changes due to the helical configuration (i.e., sloped configuration) of the conduit 130 along and relative to the interior surface 172 of the casing 170.

At point C (as shown in FIG. 1A), conduit 130 is substantially in full contact with second zone 114 of roller 110c, resulting in conduit 130 being substantially fully occluded. Moreover, at point D, conduit 130 is substantially in full contact with the second zone 114 of roller 110c, resulting in conduit 130 being substantially fully occluded. Thus, fluid is captured in conduit 130 between the rollers, such as 110b and roller 110c. As the rollers 110a, 110b, 110c continue to travel around casing 170, the rollers 110a, 110b, 110c peristaltically transport material (e.g., a fluid, slurry, or other flowable material) through conduit 130 and out of output end E. At output end E, the conduit 130 exits the casing 150. Consequently, the rollers lose contact with the conduit 130, thereby removing the occlusions they respectively form within the conduit 130. In the illustrated implementation, for example, further rotation of roller 110c will cause the second zone 114 thereof to lose contact with conduit 130, and the conduit 130 will decompress, opening the passage formed therethrough. Thus, roller 110b may expel the fluid that was captured between roller 110b and roller 110c as roller 110b continues to rotate around the interior surface 172 of the casing 170.

Pump system 100 has a variety of features. For example, the tapered portion (i.e., first zone 112) of the rollers 110a, 110b, 110c allow the conduit 130 to be compressed in a gradual manner. This reduces pulsations in the material flow through the conduit 130. This reduction in pulsations is advantageous when pump system 100 is used, for example, in a handheld ocular aspiration tool as it can improve the followability of intra-ocular aspiration fluids, minimizing the time required to perform a procedure. As another example, the rollers having a tapered portion may require little, if any, surface lubrication to function appropriately, thus reducing the operational torque required in a drive motor, as well as eliminating the need to create a lubricating bath. As an additional example, because the conduit 130 extends along a helical guide groove, the overall length of the pump system is reduced. A reduced size is especially advantageous when pump system 100 is used, for example, in a handheld ocular aspiration tool, as such a configuration enhances ease of use.

Although FIGS. 1A and 1B illustrates an example peristaltic pump 100, other peristaltic pumps may include fewer, additional, and/or a different arrangement of components. For example, a pump system may include additional rollers. As another example, a pump system may include more coils of the conduit around the casing. As a further example, a pump system may include multiple conduits in a helical pattern. In implementations having multiple helically configured flexible conduits, variable radius rollers are operable to simultaneously engage the plurality of conduits as the armature is rotated. Thus, with multiple conduits, a pump system is operable to increase a fluid output flow rate.

Other implementations are also possible. For example, another example pump may include a length of flexible conduit that forms less than a complete coil. In such implementations, the pump includes at least two rollers engaged with a fully compressing the length of conduit at any one time in order to peristaltically transport fluid. In some instances, the length of conduit may form one half of a coil. In other implementations, the pump may include a length of conduit that forms more than one half of a coil, while other pumps may have a length of conduit that forms less than one half of a coil.

FIGS. 2A, 2B, 2C, and 2D illustrate an example pump assembly 200 for use with a handheld ocular aspiration tool. FIG. 2A shows a cross-sectional view of the pump assembly 200; FIG. 2B shows a perspective view of a peristaltic pump 210 that forms part of the pump assembly 200 with a partially sectioned casing 207; and FIG. 2C shows the peristaltic pump 210 with the casing 270 and flexible conduit 230 removed to show the rollers 212 and armature 214. FIG. 2D shows a perspective view of peristaltic pump 210.

The pump assembly 200 may be coupled to a hand piece, such as hand piece 205, shown in FIG. 2A. The hand piece 205 may couple to the pump assembly 200 at a port 204 formed therein. In some instances, the hand piece 205 may be a phacoemulsification hand piece. In other instances, the pump assembly 200 may be coupled, for example, to any hand piece that includes an aspiration capability. In general, pump assembly 200 includes a motor 225 and the peristaltic pump 210. In some instances, the peristaltic pump 210 may be similar to peristaltic pump 100 and operate in a similar manner thereto.

Peristaltic pump 210 includes a number of variable radius rollers. In the illustrated example, the peristaltic pump 210 includes three rollers 212, although only two are shown, as the third roller 212 is hidden due to the configuration of the peristaltic pump 210. In other implementations, the peristaltic pump 210 may include additional or fewer rollers 212. In some implementations, as few as a single roller 212 may be used. For example, a single roller 212 may be used in a peristaltic pump having a plurality of coils of the conduit 230. In the example shown, the rollers 212 include tapered end portions 201 and a constant radius portion 203. Rollers 212 are rotationally mounted to an armature 214, which is driven by motor 230. Similar to peristaltic pump 100, flexible conduit 230 is coiled about the armature and disposed between an interior surface of the casing 270 and the rollers 212. In some instances, the armature 214 may be coupled to a rotatable shaft of the motor 225.

In some instances, the motor 225 may be an electric motor. For example, the motor 225 may be a direct current (DC) motor. In other instances, the motor 225 may, for example, be a brushless electric motor. However, in still other instances, the motor 225 may be any device operable to rotate the armature 214.

As shown, the peristaltic pump 210 is disposed at an end of the motor 225. Casing 270 may be adapted to couple to a housing 227 that surrounds motor 225. For example, the housing 227 may include a collar 229 that is received into a first end 272 of the casing 270. In some implementations, motor 225 may be part of an assembly that is reusable between different surgical procedures.

Referring to FIG. 2D, peristaltic pump 210 may also include an input port 260 to provide fluid to peristaltic pump 210 and an output port 220 to expel fluid from the peristaltic pump 210. In some instances, the peristaltic pump 210 may be disposable. For example, the peristaltic pump 210 may be removed from the motor 225 and discarded after a surgical procedure. In some implementations, the casing 270 and conduit 230 may be removed, such as by being displaced longitudinally from contact with the rollers 212 and discarded.

In some instances, the casing 270 may form part of the housing 227 for the motor 235. In some instances, the casing 270 may be a removable part of the housing 227. Thus, in some instances, the casing 270 or a portion thereof may be removable so that the peristaltic pump 210 or parts thereof(e.g., the conduit 230, armature 214, and/or a part of the casing 270 itself) may be removed and discarded. Further, in still other implementations, the pump assembly 200 may be completely disposable after one or more uses or completely reusable following a use thereof.

In operation, pump assembly 200 may be removably coupled to one or more handheld devices, such as, for example, a phacoemulsification probe, irrigation-aspiration probe, or a vitrectomy probe, to provide aspiration therefor. The pump assembly 200 and the handheld device may, for example, form an integral unit. For example, as explained above, pump assembly 200 may be coupled to a phacoemulsification probe, an irrigation-aspiration probe, or a vitrectomy probe and, as combined, collectively form part of a single handheld device. In some instances, these probes may require some type of irrigation. In some implementations, irrigation may be provided by a surgical console, a bag with gravity feed, or any other appropriate technique.

Pump assembly 200 has a variety of features. Because peristaltic pump 210 is located directly in the hand piece, undesirable effects associated with aspiration flow, for example, occlusion of aspiration flow due to lodging of aspirated materials passing through the hand piece, are minimized or eliminated. Flow may become occluded, for example, from aspirated material becoming lodged in a flow path of the hand piece. In those cases where aspiration suction is generated within a remote console, surges upon clearance of the obstruction may develop due to long, flexible conduits used to supply the suction and carry away aspirated material to the remote console. In some instances, the long, flexible conduit may be six foot or more in length. Additionally, the variable radius rollers used in the peristaltic pumps described herein and used to create the peristaltic action may incorporate a tapered portion that includes a lead-in angle. With this lead-in angle of the tapered portion, the rollers gradually occlude the conduit so as to minimize pulsations in fluid flow as the rollers create the peristaltic displacement of fluid.

The close proximity of the peristaltic pump to the aspiration site (e.g., including the peristaltic pump as part of an integral, handheld hand piece); the ability to more accurately control fluid flow; as well as the ability to develop a controlled vacuum within a hand piece, helps a user to successfully complete the lens-removal process. Particularly, the reduction in pulsations due to the tapered rollers provides for improved capture and retention of particles during aspiration. This is because the vacuum generated by the peristaltic pump fluctuates less. Further, in the context of a phacoemulsification procedure, with improved capture and retention of particles, the phacoemulsification energy is communicated to the particles more effectively. With improved capture of the particles, the particles can be emulsified more quickly, thereby reducing the total time of a procedure. Accordingly, this may reduce the time required to remove larger tissue particles, and potentially reduce patient discomfort and recovery time.

Furthermore, the variable radius rollers require little, if any, surface lubrication to function appropriately, thus reducing the operational torque required by the motor to rotate the rollers relative to the flexible conduit, as well as eliminating the need to create a lubrication bath.

FIG. 3 illustrates an example of a handheld ocular aspiration tool 300. Handheld ocular aspiration tool 300 may, for example, include a pump similar to peristaltic pump 100 or peristaltic pump 210. In some implementations, handheld ocular aspiration tool 300 may include a reusable portion 310 and a disposable portion 320. The reusable portion 310 may include a motor portion 330, and the disposable portion 320 may include a pump portion 340. The motor portion 330 may include a motor similar to the motor 235. The pump portion 340 may include a peristaltic pump as described above. Disposable portion 320 may also include an input port 350 and an output port 360.

Motor portion 330 may include a housing 332 that is illustrated as being generally cylindrical in shape, which may provide for ease of manipulation by a user. Housing 332 may have other configurations in other implementations, however. Thus, in other implementations, the housing 332 may have shapes other than a cylindrical shape. Motor portion 330 also includes a motor disposed within the housing 332. In some implementations, the motor may be an electric motor, such as a DC motor. However, the disclosure is not so limited, and the motor may be any device suitable to operate the pump portion 340.

Pump portion 340 may include a housing 342 that is adapted to mate with housing 332. For example, an end 343 of the housing 342 may receive an end 333 of the housing 332. Further, in some implementations, the housing 342 and housing 332 may be coupled together with a threaded interface, a snap fit, or an interference fit. However, any other suitable connection technique may be used. Pump portion 340 may also include a peristaltic pump, which may be similar to one or more of the pumps described herein.

As explained above, disposable portion 320 may also include an input port 350 and an output port 360. In some implementations, input port 350 may be adapted to couple to a handheld device (e.g., a phacoemulsification probe or an irritation-aspiration probe) to form an integral handheld unit. In some implementations, input port 350 may, for example, be a female portion of a luer coupling.

In some implementations, disposable component 320 may fully incorporate housing 332. Alternately, the disposable component 320 may form a separate disposable piece configured to interlock with housing 332. Those having skill in the art will also appreciate that there are a variety of combinations of reusable components and disposable components, and this disclosure is intended to encompass all of these combinations.

During a typical ocular surgery (e.g., cataract lens removal), various fluids and tissue must be aspirated from an eye. For example, natural eye fluids (e.g., aqueous humor) and supplied irrigation fluids (e.g., a water/saline solution) may be aspirated. Moreover, portions of a lens that has been emulsified as well as other materials, such as cortical material, may need to be removed in a cataract lens removal. Ocular aspiration tool 300 may be used to aspirate such fluid and tissue.

Hand held devices of the types described herein that integrate a pump of the types described herein, i.e., entirely housed on a hand held device, provides numerous benefits. Such devices provide a rapid response to a post-occlusion surge. The devices also reduce the effects of a post-occlusion surge associated with dislodging of material in an aspiration path. Particularly, the devices reduce an amount of fluid aspirated during post-occlusion surge. This reduction is achieved due to the close proximity and short length of flexible conduit within the device. The motors of these devices may be run at variable speeds to control flow rates through the pumps.

Close proximity of the pumps within these hand held devices also provide for rapid adjustments to fluctuations in vacuum. These devices also provide for improved capture and retention of particles as a result of reduced fluid flow rate pulsations or reductions in the magnitudes of those pulsations as a result of variable radius rollers. The fast vacuum and release response are due to a shorter column of fluid between the tip and the pump, as compared to a pump in a console. Thus, post-occlusion surge, i.e., a surge of fluid from the eye due to removal of an occlusion within the aspiration flow path, is reduced. These features protect the anterior capsule's integrity and improve the followability of debris suspended in the irrigation solution.

Further advantageously, these devices can reduce the cost and assembly of aspiration fluidics cassettes by allowing for thin-walled tubing instead of pressure controlled tubing, and may simplify the manufacture of dual-tube irrigation/aspiration configurations.

FIG. 4 illustrates an example irrigation-aspiration (“I/A”) unit 400. The I/A unit 400 may, for example, be used with a pump unit like pump portion 340.

The example I/A unit 400 includes a housing 410 defining a channel 414, an insert 411 received within the channel 414, a male luer fitting 419 received in a recess 421 formed at a proximal end 430 of the housing 410, and a sleeve 420 coupled to a distal end 442 of the housing 410. The housing 410 may be formed from a rigid material. For example, the housing 410 may be formed from a rigid plastic, metal or other suitable material. The housing 410 also includes an infusion port 412 that defines a channel 444.

In some implementations, male luer fitting 419 is composed of a polymer. In other implementations, male luer fitting 419 may be composed of a metal or any other appropriate material.

The insert 411 defines a channel 417 extending therethrough. The male luer fitting 419 defines a channel 436. The I/A unit 400 also includes a cannula 424 extending from a distal end 432 of the insert 411. A proximal end 434 of the cannula 424 is received in the channel 417 at the distal end 432. The cannula 424 defines a channel 425 extending therethrough. The channels 417, 425 and 436 communicate with each other to define an aspiration passage 438.

The sleeve 420 defines a channel 422. The distal end of the housing 410 may be received into the channel 422, such that the sleeve 420 expands over the distal end of the housing 410 to form a sealed interface. In other implementations, other engagements (e.g., threaded or barbed) may be used. The cannula 424 extends through the channel 422 and such that a distal end 426 of the cannula 424 extends past a distal end 442 of the sleeve 420. In some instances, the cannula 424 may be formed entirely or in part from a plastic material. In other implementations, the cannula 424 may be formed from a metal, such as stainless steel or titanium. In other instances, the cannula 424 may be formed from any suitable material. Further, in some instances, the cannula 424 may include a tip 431. The tip 431 may be utilized, for example, to polish the capsular bag. In some instances, the tip 431 may be an integral part of the cannula 424. For example, where the cannula 424 is formed from a plastic, the tip 431 may be an integral part thereof. In implementations where the cannula 424 is formed from a metal, the tip 431 may be formed from a plastic applied to the distal end 426 of the cannula 424. For example, the tip 431 may be overmolded onto the cannula 424.

An outer surface of insert 411 and an inner surface of the housing 411 define an annular space extending 440 through the housing 410. The annular space 440 is isolated from the aspiration passage 438. The annular space 440 communicates with channels 422, 444 to define an infusion passage 446. Communication between the channel 422 and the annular space 438 may be accomplished by spaces between axially extending protrusions 450 formed on the distal end of the insert 411. The infusion passage 446 is fluidly separate from the aspiration passage 438.

Infusion fluid, such as a water/saline solution (e.g., a balanced salt solution), is introduced into the infusion passage 446 via the infusion port 412. In some implementations, the infusion fluid exits the I/A unit 400 at the distal end 442 of the sleeve 420, as indicated by arrows 423. In other implementations, the sleeve 420 may include one or more ports 443 formed at the distal end 442 thereof, which permit outflow of the irrigation fluid. The infusion fluid may be provided to an eye during a procedure, such as an eye cleaning or polishing process. The infusion fluid may, for example, be provided by a surgical console.

Aspirated materials, represented by arrow 427, are drawn into the aspiration passage 438 of the I/A unit 400 via a distal opening 448. The aspirated materials pass through the aspiration passage 438 and exit the I/A unit 400 via the male luer fitting 419.

In operation, I/A unit 400 may be coupled to a pump assembly similar to a type described herein, such as pump assembly 200 or aspiration tool 300. The pump assembly may include a peristaltic pump. For example, the pump assembly may include a peristaltic pump such as peristaltic pumps 100, 200. For example, the I/A unit 400 may be coupled to a handheld pump unit at the male luer fitting 419. The I/A unit 400 may also be coupled to an irrigation supply line (e.g., from a surgical console). For example, the I/A unit 400 may be coupled to an irrigation supply line at the infusion port 412. Tip 431 may be inserted into the eye through an existing incision. Material within the eye may be aspirated while leaving the posterior capsule intact. For example, materials such as ocular tissues (e.g., cortical material and epithelial cells) may be aspirated. Simultaneously, fluids may be irrigated into the eye to stabilize it. Additionally, if desired, the posterior capsule of the eye may be polished with tip 431.

The I/A unit 400 has a variety of features. For example, by locating a pump unit nearer to I/A unit 400, chamber stability may be improved. Additionally, the I/A unit 400 may be used with a conventional surgical console if desired. A handheld pump unit can also be positioned remotely from the irrigation-aspiration unit (e.g., for ergonomic reasons) and coupled to the irrigation-aspiration unit via aspiration tubing.

Although FIG. 4 illustrates an example I/A unit 400, other systems may use other L/A units that may include fewer, additional, and/or a different arrangement of components.

The various implementations discussed and mentioned herein have been used for illustrative purposes only. The implementations were chosen and described in order to explain the principles of the disclosure and the practical application and to allow those of skill in the art to understand the disclosure for various implementations with various modifications as are suited to the particular use contemplated. Thus, the actual physical configuration of components may vary. For example, the mentioned size(s) of components and their illustrated sizing relative to each other may vary based on application. Moreover, the shapes of one or more components may vary depending on application. Thus, the illustrative implementations should not be construed as defining the only physical size, shape, and relationship of components.

Various systems and techniques for ocular surgery have been discussed, and several others have been mentioned or suggested. However, those skilled in the art will readily recognize that a variety of additions, deletions, substitutions, and modifications may be made to these systems and techniques while still achieving ocular surgery. Thus, the scope of protection should be judged based on the following claims, which may capture one or more aspects of one or more implementations.

1. A handheld ocular aspiration tool comprising:
an electrical motor;
a peristaltic pump assembly comprising:
a helically wound compressible conduit; and
at least one variable radius roller, the at least one variable radius roller configured to increasingly occlude the conduit as the roller is rotated in contact with the conduit, the at least one variable radius roller operable to substantially fully occlude the conduit at one time to pump fluid through the conduit;
a fluid input port in fluid communication with an input end of the peristaltic pump; and
a fluid output port in fluid communication with an output end of the peristaltic pump.

2. The handheld ocular aspiration tool of claim 1, wherein the at least one radius roller comprises a plurality of variable radius rollers and wherein two variable radius rollers of the plurality of variable radius rollers substantially fully occlude the conduit at one time to pump fluid through the conduit.

3. The handheld ocular aspiration tool of claim 1, wherein each of the plurality of variable radius rollers comprise a variable radius section and a substantially constant radius section.

4. The handheld ocular aspiration tool of claim 3, wherein the variable radius section is operable to gradually occlude the helically wound compressible conduit as each of the plurality of variable radius rollers is rotated in contact with the helically wound compressible conduit until the helically wound compressible conduit is substantially fully occluded, and the constant radius section is operable to maintain the occlusion as each of the plurality of variable radius rollers is continued to be rotated in contact with the helically wound compressible conduit.

5. The handheld ocular aspiration tool of claim 1, wherein the peristaltic pump assembly further comprises a casing, an inner wall of the casing providing a helical groove for receiving the helically wound compressible conduit.

6. The handheld ocular aspiration tool of claim 1, wherein the peristaltic pump assembly comprises an armature, the plurality of variable radius rollers being rotationally coupled to the armature.

7. The handheld ocular aspiration tool of claim 6, wherein the motor is operable to drive the armature.

8. The handheld ocular aspiration tool of claim 7, wherein a variable radius of the plurality of variable radius rollers has a slope of approximately 15 degrees.

9. A peristaltic pump for ocular surgery, the peristaltic pump comprising:
an armature;
a plurality of rollers rotatably coupled to the armature, each of the plurality of rollers comprising a first radius portion and a second radius portion;
a flexible conduit; and
a casing surrounding the armature and the plurality of rollers, the casing comprising a helical groove configured to receive the flexible conduit and guide the flexible conduit around the armature and the plurality of rollers, the helical groove positioning the conduit such that the flexible conduit is in a substantially open condition when contacted by the first radius portion of a roller of the plurality of rollers in a first location of the helical groove and in a substantially fully occluded condition when contacted by the second radius portion of the roller of the plurality of rollers in a second location of the helical groove.

10. The peristaltic pump of claim 9, wherein the first radius portion tapers from a first radius to a second radius larger than the first radius, wherein the first radius portion is configured to engage the conduit in a substantially unoccluded condition at the first radius and progressively occlude the conduit a location where the first radius portion engages the conduit moves from the first radius to the second radius.

11. The peristaltic pump of claim 10, wherein a gradient of the taper of the first radius portion is approximately 15 degrees.

12. The peristaltic pump of claim 10, wherein the second radius portion comprises a constant radius.

13. The peristaltic pump of claim 12, wherein the conduit is substantially fully occluded when in contact with the second radius portion.

14. The peristaltic pump of claim 1, wherein the at least one variable radius roller substantially fully occludes the conduit at at least two locations simultaneously.
